(19) United States
(12) Patent Application Publication
(10) Pub. No.: US 2023/0053073 A1
(43) Pub. Date: Feb. 16, 2023

(54) SYNERGISTIC LASER CLEANING AND WHITENING OF TEETH

(71) Applicant: Enamel Pure, Shrewsbury, MA (US)

(72) Inventors: Charles Holland Dresser, Wayland, MA (US); Nathan Monty, Shrewsbury, MA (US)

(21) Appl. No.: 17/840,654

(22) Filed: Jun. 15, 2022

Related U.S. Application Data
(60) Provisional application No. 63/226,706, filed on Jul. 28, 2021.

(51) Int. Cl. A61C 19/06; A61Q 11/00; A61K 8/22

(52) U.S. Cl. CPC A61C 19/066; A61Q 11/00; A61K 8/22

(57) ABSTRACT

Aspects relate to systems and methods for synergistic laser cleaning and whitening of teeth. In one aspect a system includes a laser system configured to perform a laser treatment on exposed tooth surfaces on a plurality of a patient's teeth, where the laser system includes a laser arrangement configured to generate a laser beam, an optical arrangement configured to direct the laser beam toward the exposed tooth surfaces, and a laser controller configured to control the laser beam to remove a pellicle from the exposed tooth surfaces, and a tray configured to apply a whitening agent to the exposed tooth surfaces after the laser treatment.

### CROSS-REFERENCE TO RELATED APPLICATIONS

This application claims the benefit of priority from Provisional Pat. App. No. 63/226,706, entitled “SYNERGISTIC LASER CLEANING AND WHITENING OF TEETH,” filed on Jul. 28, 2021, and incorporated by reference in its entirety within.

### TECHNICAL FIELD

This invention generally relates to dental systems and cosmetic treatments and, more particularly but not exclusively, to systems and methods a synergistic laser cleaning and whitening of teeth.

### BACKGROUND

Research has long showed the ability of some lasers to make dental hard tissue (e.g., enamel) less susceptible to acidic dissolution. For example, in 1998, J. Featherstone et al. demonstrated inhibition of caries progression ranging from 40% to 85% after irradiation with infrared laser sources in an article entitled “CO₂ Laser Inhibitor of Artificial Caries-Like Lesion Progression in Dental Enamel,” published in the Journal of Dental Research and incorporated herein by reference. These results have been corroborated and repeated throughout the years. Another notable project involved researchers from University of California San Francisco and Indiana University both evaluating laser treatment for caries-inhibition in different intra-oral models. The project was documented in an article entitled “Effect of Carbon Dioxide Laser Treatment on Lesion Progression in an Intraoral Model,” published in 2001 in Proc. SPIE by J. Featherstone et al. and incorporated herein by reference.

A mechanism that is believed to contribute to this inhibition of acid dissolution of laser treated hard tissue is carbonate removal. Human dental enamel is primarily (96%-wt %) comprised of hydroxyapatite (HA). Specifically, the HA found in dental enamel is non-stoichiometric carbonate-substituted hydroxyapatite (Ca₁₀(PO₄)₆₋ₓ(OH)_{2-y})(CO₃)_{x+y}, where 0≤x≤6, 0≤y≤2, which contains trace amounts of fluoride (F), sodium (Na), magnesium (Mg), zinc (Zn) and strontium (Sr)), as reported by C. Xu et al., in an article published in 2014 in J. Material Sci., entitled “The Distribution of Carbonate in Enamel and its Correlation with Structure and Mechanical Properties,” incorporated herein by reference. Xu et al. describe that increases in carbonate content within enamel correlate with decreases in mechanical properties, for example crystallinity, modulus, and hardness. It has also been long reported that increased carbonate content within enamel correlates with an increased susceptibility to acid. For example, J. Featherstone et al. reported in “Mechanism of Laser-Induced Solubility Reduction of Dental Enamel,” first published in SPIE Proc. in 1997, and incorporated herein by reference, that carbonate removal from enamel correlates to increased resistance to caries, with complete carbonate removal correlating with the optimum resistance to caries. Caries are formed through acid dissolution. Removal of carbonate within dental enamel is achieved through elevating a temperature of the enamel.

The temperature range required for removing carbonate from dental tissue has long been taught, for example by Zuerlein et al. in an article published in 1999 in Lasers in Surgery and Medicine, entitled “Modeling the Modification Depth of Carbon Dioxide Laser-Treated Dental Enamel,” incorporated herein by reference. Zuerlein et al. found that carbonate loss began when enamel reached temperatures in excess of about 400° C. during laser irradiation, but complete carbonate removal was not achieved until the enamel reached its melting point. The melting point of dental enamel is about 1280° C. as reported by Fried et al. in an article published in 1998 in Applied Surface Science entitled “IR Laser Ablation of Dental Enamel: Mechanistic Dependence on the Primary Absorber,” incorporated herein by reference. For over 20 years it has been known to the dental research community that momentarily elevating a temperature of dental enamel to a temperature in a range between about 400° C. and about 1300° C. will reduce carbonate content and increase the enamel's resistance to acid (e.g., caries and erosion).

Additional research has shown that preventative dental laser treatment can be improved upon with application of a fluoride treatment following laser treatment. For example, referring to “Non-Destructive Assessment of Inhibition of Demineralization in Dental Enamel Irradiated by a λ=9.3-μm CO₂ Laser at Ablative Irradiation Intensities with PS-OCT,” published in Lasers in Surgery and Medicine in 2008, incorporated herein by reference, A. Can et al. present a statistically significant improvement in inhibition of demineralization of dental enamel for bovine enamel surfaces treated with both laser and fluoride over bovine enamel surfaces treated with laser alone. While the results of the scientific research have shown great promise for over 20 years, commercialization and adoption of this technology has not occurred anywhere in the world.

### SUMMARY

A commercial impediment to the wider adoption of this technology is slow adoption of new technologies in dentistry and the concomitant modest-to-low level of enthusiasm for investment and commercialization in high tech dental products. A reason that explains the relatively slow adoption of new technologies in dentistry is the fact that most dentists run their own practices. The dentist, as the owner of the dental practice, is unwilling in many cases to expend resources for the latest technological advancement, when those resources can be used on other (often times personal) expenses. Additionally, most technological advances require a change in workflow for the dentist. As the owner of the practice, the dentist is not often compelled to change the way she works (i.e., workflow), unless she decides to do so. A desire on the part of the dentist to not fix what isn't broken explains the slow adoption of many high technology dental products. The slow adoption of new dental technologies is also recognized in the area of professional investment. For example, although there are many professional investment groups that focus on medical devices, there are none in the United States that focus on dental devices. A mixed cause-and-result of this environment is that few new high-tech solutions reach and penetrate the dental market.

This commercial impediment is amplified by the reality that few people really prioritize their oral health. J.P. Morgan famously quipped that “A man always has two reasons for doing anything: a good reason and the real reason.” So, it is with oral health. Seldom is oral health the real reason, people take care of their teeth. Rory Sutherland, in Alchemy, published in 2019, extrapolates that the huge benefits in oral health attained by fluoride toothpaste were achieved, not because individuals valued oral health, but because they valued good smelling breath.
“A large part of why we clean our teeth I would argue is actually fear of bad breath and vanity, in the sense that would you clean your teeth after a meal at lunchtime when you're out at a restaurant? Almost certainly not. Would you clean your teeth before a date, or first thing in the morning before you go to work? Almost certainly yes. Further bit of evidence. 95% of the world's toothpaste seems to be flavored with mint, which doesn't make sense from a dental health perspective, but makes a hell of a lot of sense from a bad breath perspective.”

It would seem that the most important oral health activity (brushing one's teeth) is motivated by both a good reason—improved oral health and a real reason—improved smelling breath. While science shows without equivocation that a good reason exists for preventive dental laser treatment, there has been no real reason for the dental market to embrace it.

Systems and methods for preventative dental laser treatment have been known to science for decades. However, the known state-of-the-art (including all of the above-mentioned references) fail to teach a way for the treatments to be made attractive to a dental market that is notoriously slow to adopt high tech solutions and a larger public whose interest in oral health is largely confined to fleeting moments sitting in the dentist's chair. In order for dental patients to benefit from decades of scientific breakthroughs in preventative dental laser treatments, laser systems and methods must be developed that are adopted by dental offices and desirable to the dental market.

Preventative laser treatment technology has been known by researchers for almost 30 years to increase caries-resistance of treated teeth up to tenfold. * †‡. But the technology has never successfully been commercialized or improved the quality of life for a single dental patient (outside of clinical studies). The applicant believes the reasons for this are, in large part, outlined above and generally relate to motivations of industry stakeholders (e.g., dentists, patients, and businessmen) and to the dental industry environment in general. To bring this promising technology to market, aspects of the present invention relate not only to new technical variations for methods and systems of treatment, but applications of this technology to treat teeth in a manner never before attempted, but expected to be widely popular. At least for this reason, embodiments of the present invention relate to improving the rate of teeth whitening by first removing pellicle from the surface of the teeth with a laser. Just as Rory Sutherland has pointed out that many people are seemingly motivated to brush their teeth in order to have good smelling breath and appreciate the oral benefits of brushing secondarily, the applicant submits that many patients will elect dental laser treatment to whiten and improve the aesthetic qualities of their teeth and benefit from the known anticavity benefits of laser treatment secondarily.

As mentioned above, high-tech solutions are viewed as slow to be adopted in the dental market and professional investors eschew the opportunity to invest in high-tech dental solutions. However, an exception exists in the aesthetic dental market. Countless laser, white light, and UV whitening devices have been developed and entered into the dental market in the past 10 years. Although, the performance of these systems is modest and in some cases the claims of these systems are exaggerated, their presence in the market demonstrates an acceptance for high-tech dental solutions that promise aesthetically pleasing outcomes. University research exclusively describes preventative CO₂ laser treatment as an anti-cavity procedure, which is applied to small at-risk portions of teeth (e.g., pits and fissures). However, embodiments of the present invention relate to systems and methods that can be used to aid in whitening teeth.

Commonly, at every dental cleaning the patient is treated with scaling of the dental hard tissue with metal picks (e.g., scalers and explorers), as well as dental prophylaxis. The reason for these procedures is, in part, to remove biofilm (e.g., tartar and plaque) that builds up on teeth. Exemplary embodiments described herein selectively remove biofilm from the teeth with touchless, sensationless laser procedure. Unlike, with the mechanical methods described above, removal of biologics using a laser leave no smear layer. The teeth after laser treatment are pristine and free from the protective pellicle that normally protects the enamel, allowing for the direct application of a synergistic whitening treatments to the exposed enamel. Direct application of the whitening composition to a pristine tooth surface in some cases, is further improved upon by use of light assisted whitening, which additionally accelerates the whitening process.

In one aspect, a system for synergistic laser cleaning and whitening of teeth includes a laser system configured to perform a laser treatment on exposed tooth surfaces on a plurality of a patient's teeth, where the laser system includes a laser arrangement configured to generate a laser beam, an optical arrangement configured to direct the laser beam toward the exposed tooth surfaces, and a laser controller configured to control the laser beam to remove a pellicle from the exposed tooth surfaces, and a tray configured to apply a whitening agent to the exposed tooth surfaces after the laser treatment.

In another aspect, a method of synergistic laser cleaning and whitening of teeth includes performing, using a laser system, a laser treatment on exposed tooth surfaces on a plurality of a patient's teeth, where the laser treatment includes generating, using a laser arrangement, a laser beam, directing, using an optical arrangement, the laser beam toward the exposed tooth surfaces, and controlling, using a laser controller, the laser beam to remove a pellicle from the exposed tooth surfaces, applying, using a tray, a whitening agent to the exposed tooth surfaces within a certain post-laser time after the laser treatment, such that the composition directly wets the exposed tooth surfaces, after the pellicle has been removed, and maintaining, using the tray, application of the whitening agent to the exposed tooth surfaces for a prescribed whitening duration.

Any combination and permutation of embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1A is an isometric view of an exemplary whitening tray;

FIG. 1B is a front view of an exemplary whitening tray;

FIG. 1C is a vertical cross-sectional view of an exemplary whitening tray;

FIG. 1D is a horizontal cross-sectional view of an exemplary whitening tray;

FIG. 2 is a graph providing an exemplary dose-response curve showing an Arndt-Schulz curve representing a biphasic dose response;

FIG. 3 is a schematic illustration of a laser beam being used to remove a biofilm on a dental surface, according to certain exemplary embodiments;

FIG. 4 is an illustration of an exemplary system for dental laser treatment;

FIG. 5 is a flow diagram of an exemplary method of synergistic laser cleaning and whitening of teeth; and

FIG. 6 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, a complete software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

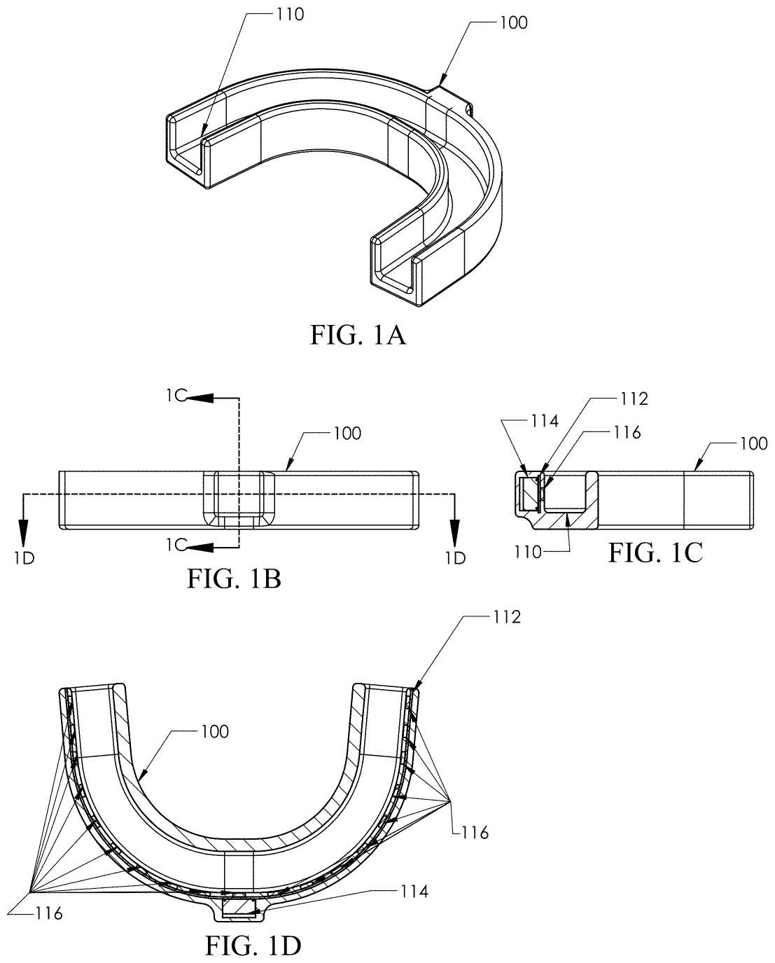

FIG. 1A illustrates an isomeric view of an exemplary tray 100 for applying a whitening agent. In some embodiments, the tray 100 is shaped to fit over an upper arch or a lower arch, such that a whitening agent placed within a channel 110 of the tray 100 at least partially covers some exposed portion of teeth within the arch. In some cases, the tray 100 and channel 110 are custom formed to fit over an individual's arch. Alternatively, in some cases, the tray 100 and channel 110 are non-custom, for example so that a single tray size may fit over any number of patient's arches. The tray 100, in some embodiments, is manufactured by way of molding. In some cases, the tray 100 comprises a polymer, such as without limitation polyethylene (PE), silicone, and the like. In some cases, the tray 100 comprises a polymer that is substantially transparent to at least a portion of a visible wavelength of light. For example, in some cases, the tray 100 is generally clear (i.e., transparent).

FIG. 1B illustrates a front view of the tray 100 with cross-section indicators 1C-1C and 1D-1D. A vertical cross-sectional view is shown in FIG. 1C. A horizontal cross-sectional view is shown in FIG. 1D.

Referring now to FIG. 1C and FIG. 1D, a vertical cross-sectional view and a horizontal cross-sectional view of the tray 100 is shown. The tray includes circuitry 112. Exemplary circuitry 112 can include printed circuit boards, flex circuitry, wiring, cables, and the like. In some cases, the circuitry 112 provides electrical communication with one or more elements, for example an energy source 114 and a light source 116. In some cases, the circuitry 112 includes a controller configured to control the light source 116. In some cases, the circuitry 112 is additionally in electrical communication with an energy source 114. Exemplary energy sources include batteries, an alternating current to direct current converter, a power supply and the like. In some cases, the energy source 114 includes a rechargeable battery, for example without limitation a lithium-ion battery. Alternatively or additionally, in some cases, the energy source 114 includes a disposable (i.e., sing-use) battery. An exemplary single-use battery includes without limitation a zinc-air battery.

In some embodiments, energy source 114 may include a metal-air battery, such as without limitation a zinc-air battery. A metal-air battery is an electrochemical cell that uses an anode made from pure metal and an external cathode of ambient air, often with an aqueous or aprotic electrolyte. As a metal-air battery discharges, a reduction reaction occurs in the ambient air cathode while the metal anode is oxidized. In many cases, specific capacity and energy density of metal—air electrochemical cells are higher than that of lithium-ion batteries. In some cases, zinc-air batteries are metal—air batteries powered by oxidizing zinc with oxygen from air. These batteries have high energy densities and are relatively inexpensive to produce. Sizes range from very small button cells, such as without limitation those for hearing aids, larger batteries, such as those used in film cameras, to very large batteries, such as those used for electric vehicle propulsion and grid-scale energy storage.

In some embodiments, during discharge of a zinc-air battery, a mass of zinc particles forms a porous anode, which is saturated with an electrolyte. In some cases, oxygen from the air reacts at a cathode and forms hydroxyl ions which migrate into the zinc paste and form zincate (Zn(OH)₄²⁻), releasing electrons to travel to the cathode; in some cases, as the zincate decays into zinc oxide, water returns to the electrolyte. In some embodiments, the water and hydroxyl from the anode are recycled at the cathode, so the water need not be consumed. In some cases, zinc-air battery reactions produce a theoretical 1.65 volts, however in practice zinc-air batteries achieve about 1.25-1.5 volts.

With continued reference to FIG. 1, tray 100 includes circuitry 112, which in some embodiments, which may include or otherwise be communicative with a computing device. A computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card, Bluetooth interface), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented using a “shared nothing” architecture in which data is cached at the worker, in an embodiment, this may enable scalability of system 100 and/or computing device.

With continued reference to FIG. 1, in some embodiments, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

In some embodiments, the tray 100 includes a light source, 116. The light source 116, in some cases, includes one or more light emitting diodes 116. Alternatively, the lights source 116 can include any source of light, for example a laser, a light emitting capacitor, any coherent light source or non-coherent light source. In some cases, the light source 116 has a wavelength within at least one specified range. Exemplary non-limiting wavelength ranges including UV wavelengths, visible wavelengths, near-infrared wavelengths, infrared wavelengths, and the like. In some cases, the light source 116 has wavelength within a photoactivating wavelength range, believed to expedite whitening, for example a wavelength having a shorter high-energy wavelength, such as without limitation UV and/or blue wavelength for instance wavelengths below about 500 nm and above about 100 nm. An exemplary non-limiting light source include a 450 nm wavelength LED, e.g., Thorlabs Part No. LEDS450, from Thorlabs of Newton, N.J. The LEDS450 has a center wavelength of 450 nm, a bandwidth of 13 nm (FWHM), a viewing half angle of 46°, a DC forward current (Maximum) of 180 mA, a forward voltage (Maximum) of 6.6V, an emitter size of 1 mm×0.5 mm, two emitters, and an expected minimum lifetime greater than 25,000 hours.

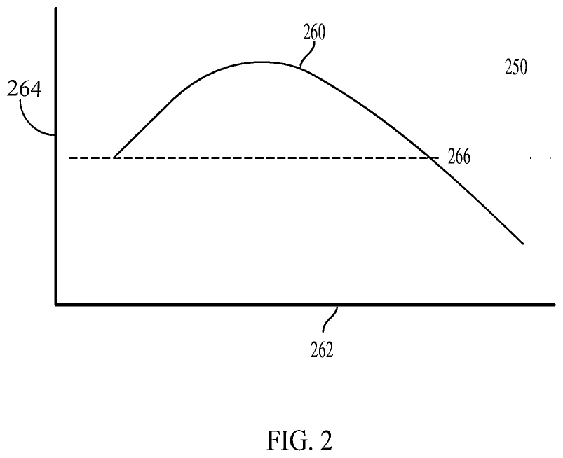

In some embodiments, light source 116 has a wavelength within a photobiomodulation (PBM) wavelength range. In some cases, the PBM wavelength range include one or more of ultraviolet, visible, and/or infrared light. In some cases, light source 116 illuminates a PBM wavelength with a prescribed PBM dosage. In some case, PBM has a multi-phasic (e.g., biphasic) dosage response, which is explained in greater detail in reference to FIG. 2 below. As a result, a prescribed PBM dosage may be an accumulated energy dosage (e.g., in Joules), a power dosage (e.g., in Watts), in time (e.g., in Seconds), or in any combination of the preceding measures.

Photobiomodulation, which is at times referred to as low level light (or laser) therapy (LLLT), can act according to a biphasic (i.e., hormesis) dose-response relationship. Hormesis dose-responses can be understood as following the Arndt-Scholz rule of pharmacology: “For every substance, small doses stimulate, moderate doses inhibit, and large doses kill.” Thus, a hormesis dose-response can be modeled using an Arndt-Schulz curve. FIG. 2 shows an exemplary dose-response graph 250 having an Arndt-Schulz curve 260 according to an exemplary embodiment of the present disclosure. A horizontal axis 262 indicates an exemplary dose, and a vertical axis 264 indicates an exemplary response. A dashed horizontal line 266 provides a control response indicating no dosage applied. For example, small dosages introduce an increasingly positive response, up to, e.g., a maximum at which increasing dosage reduces the response. Further increasing dosage can inhibit the exemplary response as the dose-response curve 260 falls below the control line 266. Huang, Sharma, Carroll, and Hamblin's 2011 publication entitled “Biphasic Dose Response in Low Level Light Therapy” published in Dose-Response, and incorporated herein by reference, describes research showing hormesis in LLLT dosage. The Huang publication enumerates 22 LLLT studies, including in vitro, animal, and clinical studies, which have shown hormesis. This publication demonstrates the difficulty biphasic (or even triphasic) dose responses can present for researchers as well as clinicians. As scientists and doctors are reporting on difficulties with the biphasic dosing of photobiomodulation, such a device made available for use directly to a user, or in clinical settings, should carefully control light dosage so as to prevent or at least reduce over-dosing and the possible resulting inhibitory effects.

Exemplary PBM wavelengths are enumerated in a table below:

| Representative | PBM Wave- | List of Narrowband List |
|---|---|---|
| PBM Wave- | length Range | Nom. Center Wavelength |
| length (nm) | (nm) | (nm) |
| 665 | 600-700 | 600, 610, 620, 635, 640, 643, |
| | | 650, 660, and 680 |
| 765 | 700-800 | 720, 730, 740, 765, and 770 |
| 850 | 800-900 | 810, 820, 830, 850, 855, 856, |
| | | 870, 875, 880, 885, 890, and |
| | | 900 |

Exemplary PBM light dosages are enumerated below:

| Nom. | Min. | Nom. | Max | Min | Nom. | Mas | Min | Nom. | Max |
|---|---|---|---|---|---|---|---|---|---|
| Wavelength | Irradiance | Irradiance | Irradiance | Fluence | Fluence | Fluence | Time | Time | Time |
| (nm) | (W/cm²) | (W/cm²) | (W/cm²) | (J/cm²) | (J/cm²) | (J/cm²) | (S) | (S) | (S) |
| 665 | 0.0005 | 0.008 | 5 | 0.5 | 5 | 50 | 60 | 600 | 14400 |
| 765 | 0.0005 | 0.008 | 10 | 0.5 | 5 | 100 | 60 | 600 | 14400 |
| 850 | 0.001 | 0.008 | 10 | 1 | 5 | 100 | 60 | 600 | 14400 |

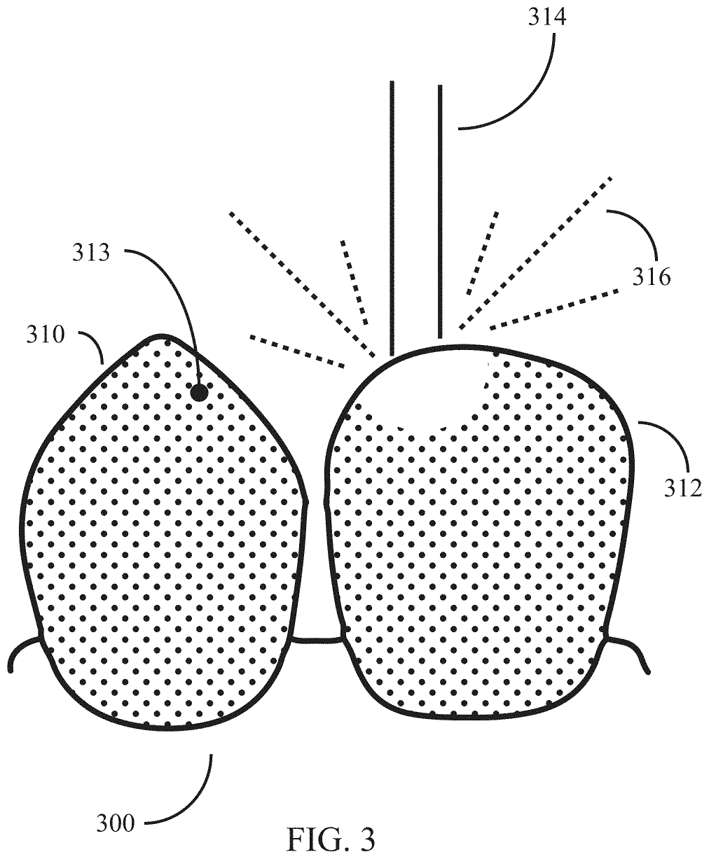

Referring to FIG. 3, a schematic illustration of a mouth 300 having a first tooth 310 and second tooth 312. The first tooth 310 is completely covered by a biofilm 313. The second tooth 312 is shown having the biofilm 313 partially removed by a laser beam 314. The laser beam 314 is directed toward a surface of the second tooth 312. The laser beam 314 heats a portion of the surface of the second tooth 312. Heating of the surface of the second tooth causes the biofilm 313 to be removed. In some cases, the biofilm is removed by way of sublimation, vaporization, thermal denaturization, photonic denaturization, or laser ablation. In some cases, the biofilm is moderately well absorbed by the laser beam (e.g., has an absorption coefficient greater than about 100 cm⁻¹) and heating of the biofilm at least partially contributes to removal of the biofilm. The removed biofilm (e.g., biofilm particulate and/or vapors) 316 is shown being ablated away from the laser heated surface, in FIG. 3.

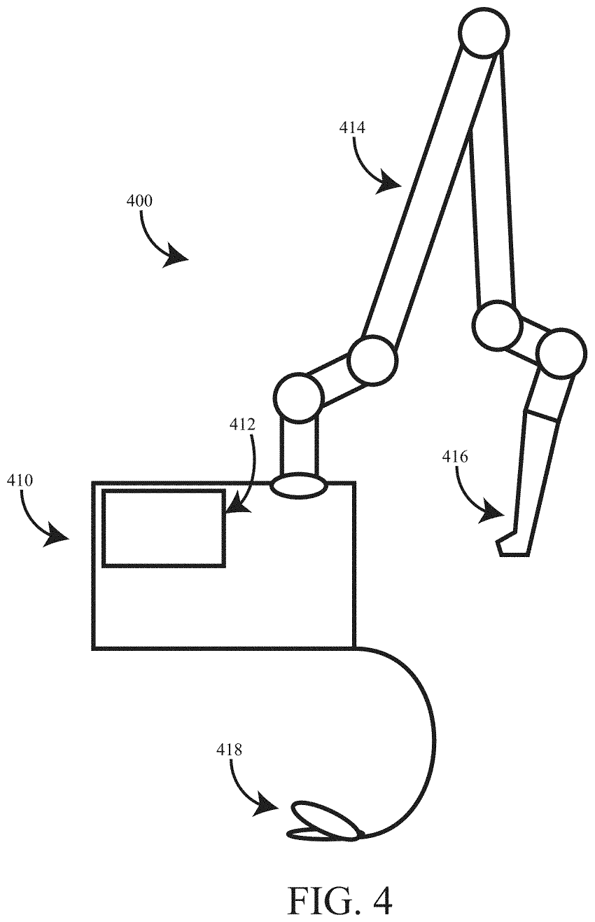

An exemplary system 400 is shown in FIG. 4 The system 400 includes a console 410. The console 410 houses components of the system 400, for example, a laser source to generate the laser beam, a direct current (DC) power supply to power the laser source, a beam shaper to shape an energy profile of the laser beam, a compressed air system to deliver compressed air for bulk cooling of dental hard tissue being treated, and a user interface 412 for user control. A beam delivery system 414 directs the laser beam to a hand piece 416. Exemplary beam delivery systems 414 include articulated arms, waveguides, and fiber optics. An exemplary articulated arm is provided by Laser Mechanisms of Novi, Mich., U.S.A. The hand piece 416 is configured to be used intra-orally (i.e., within an oral cavity). Typically, the hand piece 416 includes a focus optic (not shown) that converges the laser beam to a focal region outside of the hand piece 416. In accordance with one embodiment, the system 400 is operated with a foot pedal 418, which is configured to initiate the laser source.

In accordance with one embodiment, the system 400 is used by a clinician. First, the clinician inputs operating parameters into the user interface 412, for example by using a touch screen. Then the clinician places the hand piece 416 within a patient's mouth and directs the hand piece 416 toward dental hard tissue. For example, the clinician positions the hand piece 416 so that a focal region of the laser beam is coincident with or near (e.g., +/−1 mm, 2 mm, 3 mm, or 5 mm) a surface of a tooth. Then, the clinician activates the laser by stepping on a foot pedal 418. The clinician moves the hand piece 416 within the patient's mouth, carefully directing the focal region of the laser beam near every treatment surface of the patient's teeth.

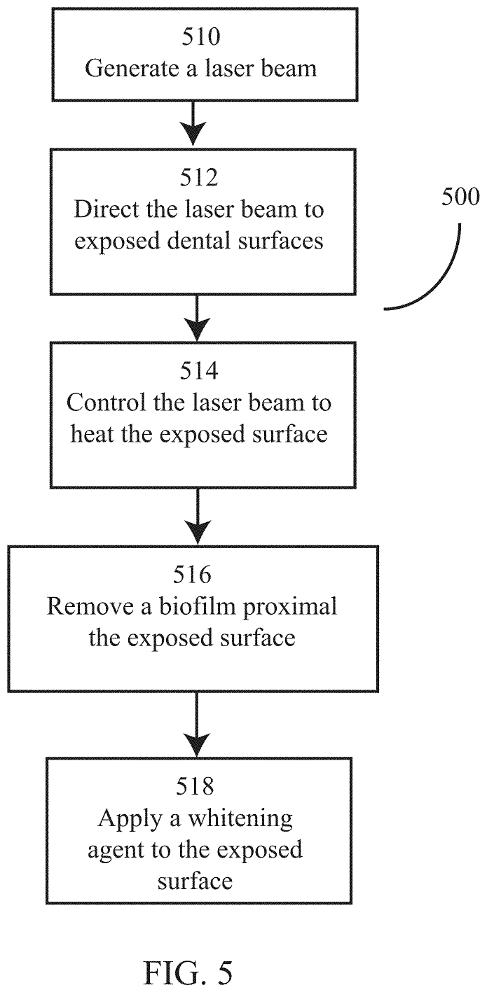

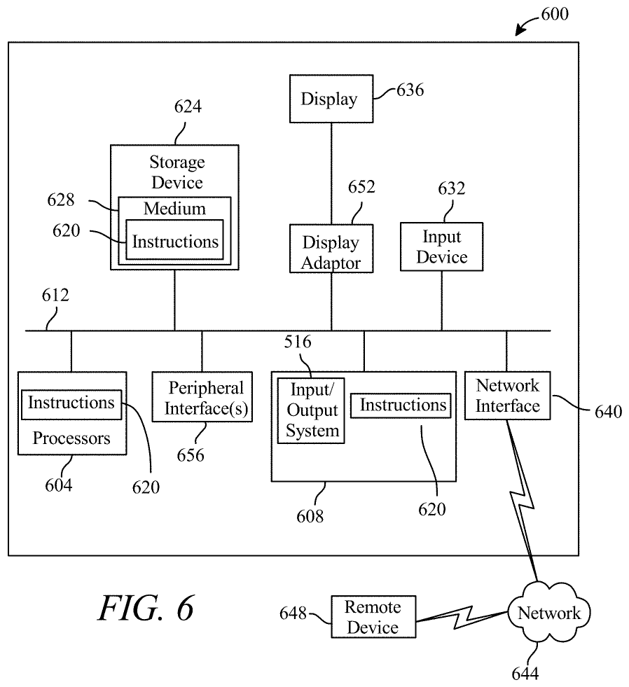

Referring to FIG. 5, a flowchart 500 is presented that describes a method of synergistic laser cleaning and whitening of teeth. First, a laser beam is generated 510. The laser beam typically has a wavelength in a range between about 8 and 12 micrometers (e.g., about 9.3 μm, about 9.6 μm, or 10.6 μm). The laser treatment begins by generating a laser beam. The laser beam is typically generated using a laser source. Exemplary laser sources include: CO₂ lasers having a wavelength between 9 μm and 11 μm, fiber lasers, diode pumped solid state lasers (DPSS), Q-switched solid-state lasers (e.g., third harmonic Nd:YAG lasers having a wavelength of about 355 nm), Excimer lasers, and diode lasers. Commonly the laser beam has a wavelength that is well absorbed (e.g., has a wavelength having an absorption coefficient greater than 1 cm⁻¹, 100 cm⁻¹, or 1,000 cm⁻¹) by a dental hard tissue. The laser beam is typically pulsed and has a pulse duration, that is about a thermal relaxation time of the enamel and laser beam (e.g., the pulse duration is no more than 10 to 100 times greater than the thermal relaxation time). In some embodiments, the laser beam is pulsed at a repetition rate that has a period (i.e., inverse of the repetition rate) that is greater than the thermal relaxation time (e.g., greater than the thermal relaxation time, greater than 10 times the thermal relaxation time, or greater than 100 times the thermal relaxation time).

Thermal relaxation time is defined, in some cases, to represent an estimated amount of time required for thermal diffusion to reduce temperature in a layer of dental tissue, having a certain thickness, by approximately one half. Commonly, the thickness of the layer of dental tissue is taken to be an optical penetration depth, which is approximated as an inverse of the absorbance coefficient of the laser radiation in dental tissue, or:

$$X ( λ ) = \frac{1}{{μ}_{a} ( λ )}$$

where, X(λ) is the optical penetration depth as a function of wavelength, A is wavelength of the laser, and μₐ(λ) is the absorption coefficient of dental tissue at the laser wavelength. The thermal relaxation time, or time for a temperature required for tissue at a certain depth to reach about 84% of a surface temperature, is approximated as:

$$t = \frac{{x}^{2}}{4 K}$$

where, t is the thermal relaxation time, x is the depth of the location of the tissue, and K is a thermal diffusivity of the tissue (e.g., enamel, dentine, or cementum). In some cases, it is appropriate to calculate an axial (depth orientated) thermal relaxation time, as described above and (by using the optical penetration depth as x). Alternatively, it is appropriate to calculate a radial thermal relaxation time that represents an amount of time for tissue radially displaced from the laser beam to heat as a result of pulsed laser cooling (by using a width of the laser beam as x). In many cases, the laser beam width is larger than the optical penetration depth and as a result the shorter of the two thermal relaxation times (axial and radial) is the axial thermal relaxation time. Thermal diffusivity is given as:

$$K = \frac{k}{ρ {c}_{p}}$$

where, K is the thermal diffusivity (for example, in units of m²/s, k is thermal conductivity (for example, in units of W/[mK]), ρ is density (for example, in units of kg/m³), and cₚ is specific heat capacity (for example, in units of J/[kgK]). Exemplary thermal parameters for dental enamel include, a density of about 2.9 g/cm³, a specific heat capacity of about 0.75 J/(g° C.), a thermal conductivity of about 9.2×10⁻³ W/(cm° C.), and a thermal diffusivity of about 0.0042 cm²/s. Exemplary thermal relaxation times for dental enamel with a 10.6 micron and 9.6 micron laser are about 1 μs and about 90 μs, respectively.

The laser beam is directed to exposed dental surfaces 512. Exposed dental surfaces comprise all tooth surfaces in the mouth, including buccal surfaces, facial surfaces, palatal surfaces, lingual surfaces, occlusal surfaces, interproximal surfaces, mesial surfaces, and distal surfaces. Generally, all tooth surfaces in the mouth have a salivary pellicle formed upon them. This pellicle layer (i.e., biofilm) is formed from proteins and glycoproteins in saliva. Under normal oral conditions, the pellicle layer protects the underlying tissue surface (i.e., enamel, dentin, or cementum). For example, the pellicle protects the dental tissue from direct exposure to acids, such as those formed by bacteria or those ingested by the patient. The pellicle is also normally colonized by bacteria (e.g., gram positive aerobic cocci, such as Streptococcus sanguins, Streptococcus mutans, and Lactobacilli). Because the pellicle covers and protects the dental surfaces, it also prevents dental surfaces from being directly exposed to any number of compositions that are being employed to bring about a desired effect. For example, some tooth whitening procedures first instruct the patient to brush her teeth with a weak acid formulation to break down the pellicle, before applying the whitening (e.g., hydrogen peroxide) composition to whiten her teeth. Brushing one's teeth with acid is not a daily routine that most would consider healthy or anti-aging for teeth, but it increases the speed of effective whitening treatment by allowing the enamel to be directly wetted by the whitening composition.

In some cases, the treatment is performed especially on aesthetic enamel surfaces (i.e., enamel surfaces that are visible when the patient smiles fully). Alternatively, in some cases, the treatment is performed on most or nearly all (e.g., greater than 50% or greater than 80%) of enamel surfaces. In some embodiments, the laser beam is directed into an intra-oral cavity using a beam delivery system. The laser beam is often directed within the intra-oral cavity using a hand piece. In some embodiments, the laser beam is converged, using a focus optic, as it is directed toward the dental hard tissue, such that it comes to a focal region proximal the surface of the dental hard tissue. Exemplary focus optics include lenses (e.g., Zinc Selenide Plano-Convex lenses having an effective focal length of 200 mm) and parabolic mirrors. In some embodiments, the laser beam is scanned as it is directed toward the surface of the dental hard tissue by a beam scanning system. Exemplary beam scanning systems include Risley prisms, spinning polygon mirrors, voice coil scanners (e.g., Part No. MR-15-30 from Optotune of Dietikon, Switzerland), galvanometers (e.g., Lightning II 2-axis scan head from Cambridge Technology of Bedford, Mass., U.S.A.), and a gantry with a translating focus optic. Scanning methods related to dental laser systems are described in U.S. Pat. No. 9,408,673 by N. Monty et al., incorporated herein by reference.

In some embodiments, a parameter of the laser beam is controlled to affect treatment. Typically, the parameter of the laser beam is controlled in order to heat a portion of the surface of the dental hard tissue to a temperature within a range, for example between about 500° C. and about 1300° C. Exemplary laser parameters include pulse energy, pulse duration, peak power, average power, repetition rate, wavelength, duty cycle, laser focal region size, laser focal region location, and laser focal region scan speed. During laser treatment a laser beam is generated and directed toward a surface of dental hard tissue. Typically, the laser beam is pulsed at a prescribed repetition rate and has a certain pulse duration. Alternatively, pulses can be delivered on demand, and the pulse duration can vary (for example, to control heating of the surface of the dental hard tissue). As a result of the irradiation of the surface, a temperature of the surface rises typically to within a range (e.g., between 400° C. and 1300° C.) momentarily (e.g., during a duration of the laser pulse) and cools back to a normal temperature range (e.g., within a range of 20° C. and 60° C.). As a result of the momentary temperature rise biological materials previously near or adhered to the surface of the dental hard tissue (e.g., pellicle, bio-film, calculus, and tartar) are at least partially removed or denatured. In some embodiments, this removal of biological materials substantially cleans the teeth and the laser treatment replaces other tooth cleaning procedures typically performed during a dental check-up (e.g., scaling and polishing). Additionally, as described above, heating the surface of the dental hard tissue removes impurities (e.g., carbonate) from the dental hard tissue and makes the dental hard tissue less-susceptible to acid dissolution (e.g., demineralization). An exemplary laser energy dosage delivered during a single treatment does not exceed an average power of about 2 W, a treatment time of about 600 seconds, and therefore does not deliver more than about 1200 J of laser energy to the oral cavity. In some embodiments, the laser treatment is performed after other treatments during a dental visit. For example, in some cases the dental laser treatment is performed only after one or more of removal of plaque and tartar (with one or more manual instruments), professional flossing, and power polishing (i.e., dental prophylaxis). This order of steps in some cases is considered advantageous, as the laser treatment purifies only an outer portion (e.g., 2 μm thick) of the dental enamel and some dental cleaning treatments can remove a portion of dental enamel (e.g., power polishing), potentially removing the enamel which has just been purified.

In some exemplary embodiments, in order to perform effective treatment, the enamel surface needs to have its temperature raised momentarily to within an elevated range (e.g., about 400° C. to about 1500° C.). As described throughout, elevating the temperature of enamel changes the chemical composition of hydroxyapatite within the enamel. Dental enamel comprises 96% (wt %) hydroxyapatite, 3% water, and 1% organic molecules (lipids and proteins). Specifically, dental enamel comprises 96% calcium-deficient carbonated hydroxyapatite (CAP), with a chemical formula approximated by Ca₁₀₋ₓNaₓ(PO₄)_{6-y}(CO₃)_{z}(OH)₂₋ᵤFᵤ. The ideal chemical formula for hydroxyapatite (HAP), by comparison, is approximated as Ca₁₀(PO₄)₆(OH)₂. The calcium deficiency of dental enamel is shown by the x in Ca₁₀₋ₓ. Some of the calcium is replaced by metals, such as sodium, magnesium, and potassium. These metals together total about 1% of enamel. Some of the OH molecules in dental enamel are replaced by F. But, the major difference between CAP and HAP comes with the presence of carbonate. Carbonate comprises between about 2 and about 5% (wt %) of dental enamel. The presence of carbonate within the hydroxyapatite structure disturbs a crystal lattice of the CAP, changing the size and shape of the unit crystal form and resulting in different mechanical and chemical properties between CAP and HAP. Increased carbonate content in enamel correlates with increases in susceptibility to acid and inversely correlates with crystallinity, hardness, and modulus (i.e., stiffness). Said another way the purer HAP erodes (through acid dissolution), wears (through mechanical means), and ages more slowly, compared to CAP.

As has been described in literature, including the Co-owned Int. Patent Appl. No. PCT/US21/15567, entitled “Preventative Dental Hard Tissue Laser Treatment Systems, Methods, and Computer-Readable Media”, by C. Dresser et al., incorporated herein by reference, carbonate can be removed from dental enamel by laser irradiation at prescribed parameters. Specifically, by using a laser source that is well absorbed (e.g., absorbance of at least 500 cm⁻¹) in dental enamel, and heating the surface of the tooth momentarily (e.g., at pulse durations that are no greater than 100× a thermal relaxation time) to a temperature of at least about 400° C., carbonate is driven (e.g., sublimated) from the enamel.

The laser beam, as it is directed to the exposed dental surface, is typically better absorbed by the underlying dental surface (e.g., enamel) than by the pellicle layer. For example, an absorption coefficient of a CO₂ laser beam in enamel is 8,000 cm⁻¹, 5,500 cm⁻¹, and 825 cm⁻¹ for 9.3 μm, 9.6 μm, and 10.6 μm wavelengths respectively. § ** For the same 9.3 μm, 9.6 μm, and 10.6 μm wavelengths the absorption coefficient in 100% water is about 600 cm⁻¹ and in 4% water is about 30 cm⁻¹. †† Depending upon a hydration level of the salivary pellicle a nominal to moderate amount of absorption of laser radiation will occur within the pellicle. Conversely, a moderate to very high level of laser radiation occurs within the enamel. In cases of high laser absorption (e.g., absorption coefficient greater than 600 cm⁻¹) in enamel and low laser absorption (e.g., absorption coefficient less than 600 cm⁻¹) in the pellicle, most of the laser energy is absorbed in a surface of the enamel. The laser energy absorbed in the outer surface of the enamel typically occurs in such a narrow width (the width of the laser beam) (e.g., less than 2 mm, less than 1 mm, less than 0.5 mm or less than 0.25 mm) and at such a thin depth (approximated by an optical penetration depth, which is an inverse of the absorption coefficient) (e.g., less than 0.2 mm, less than 0.1 mm, less than 0.02 mm, less than 0.01 mm, less than 0.005 mm, or less than 0.002 mm) that a small amount of energy (e.g., less than 100 mJ, less than 50 mJ, less than 20 mJ, less than 10 mJ, less than 5 mJ, or less than 2 mJ) raises a temperature of the surface of the enamel significantly (e.g., greater than 50° C., greater than 100° C., greater than 200° C., greater than 500° C., greater than 700° C., or greater than 1000° C.) momentarily (e.g., less than 10 ms, less than 1 ms, less than 0.5 ms, or less than 0.1 ms). As a result of this momentary rise of enamel surface temperature, the pellicle layer is removed (e.g., vaporized, sublimated, ablated, or denatured). Unlike other forms of removing the pellicle layer (e.g., dental prophylaxis, acid etching, or abrasion), laser treatment does not risk removal of the enamel, but substantially only the pellicle layer, along with any plaque, tartar or surface contaminants (i.e., biofilm), is removed. In some embodiments, the enamel is raised to a temperature within a first range between about 100° C. and about 400° C. In this first range, the salivary pellicle is substantially removed, but the enamel does not experience any substantial improvements to its mechanical properties (e.g., removal of carbonate, increased crystallinity, increased modulus [i.e., stiffness], increased resistance to acid or increased hardness). In some cases, heating the enamel surface within this first range is advantageous as the pellicle is removed, while the underlying enamel remains receptive to topical compositions (e.g., whiting agents, remineralization agents, and fluoride treatments). Alternatively, in some embodiments, the enamel is raised to a temperature within a second range between about 400° C. and about 1500° C. When heated to a temperature within this second range, the surface of the enamel has the pellicle layer removed and also experiences improvements to its mechanical properties (e.g., removal of carbonate, increased crystallinity, increased modulus [i.e., stiffness], increased resistance to acid, or increased hardness). Heating of enamel to a temperature within this range removes carbonate impurities from within the enamel surface. ‡‡ A lack of carbonate impurities within enamel (i.e., hydroxyapatite) correlates with an increase in mechanical properties, such as crystallinity, modulus, hardness, and resistance to acid. §§ ***

In some embodiments, one or more laser parameters are controlled to control the temperature rise of the dental surface. Exemplary parameters that can be controlled to affect the temperature rise include, pulse duration, pulse energy, repetition rate, fluence, irradiance, peak power, average power, number of overlapping pulses at a given location, and time between pulses (i.e., repletion period). The fluence of the laser at the surface of the enamel is commonly selected to affect a temperature rise of dental enamel. For example, with a 9.3 micron laser and a pulse duration in a range between about 0.1 and about 100 μs, a fluence greater than about 0.5 J/cm′ and less than about 5 J/cm² causes elevation of enamel surface temperature to within the 400° C. to 1500° C. range. Predictive modeling of effects of laser treatment on surface temperature rise has been found substantially accurate. For example, a nodal finite element analysis using Fourier conduction, Beer's absorption, and Newton's cooling with known parameters has been performed by the applicant. This analysis demonstrated that predictable surface temperature results are attained through use of the model. The model was verified by bench tests with multiple laser sources having peak powers ranging from about 50 W to about 1000 W. Further disclosure related to parameter selection for dental surface temperature rise and nodal-FE analysis for parameter selection is described in detail in U.S. Patent Appl. No. 62/968,910, entitled Laser Delivery of Transverse Electromagnetic Modes for Even Preventative Dental Hard Tissue Treatment, by N. Monty et al., incorporated herein by reference. Predictable temperature rise based upon known thermal and photonic constants allows for the selection and control of parameters to control temperature rise. For example, in some embodiments, a laser parameter is controlled in order to control the temperature rise of a non-enamel dental hard tissue (e.g., dentin, cementum, or osseous tissue) to a range having a lower boundary and an upper boundary. The lower boundary being selected to exceed a denaturing threshold of the biofilm (e.g., at least 50° C. or at least 100° C.). The upper boundary being selected not to exceed a tissue combustion, carbonization, incineration, or melting threshold (e.g., no more than about 200° C., no more than about 400° C., no more than about 600° C., or no more than about 1000° C.).

In some cases, removal of the biofilm completes the laser procedure. Alternatively, in some embodiments, a composition is applied directly to the surface 518, with substantially no pellicle (or biofilm) layer between the composition and the surface. In some embodiments, the direct application of the composition without an intermediary pellicle or biofilm layer improves the efficacy of the composition and, in some cases, allows a decreased dosage (e.g., concentration of active ingredient) of the composition to be used. Exemplary compositions include whiting agents, fluoride treatments, desensitizing agents, remineralization agents, sealants, composite filings, etches, wetting agents, and adhesives.

In some cases, the composition includes a whitening agent. As described above whitening agents are reduced in efficacy by the protective salivary pellicle, which prevents all of the oxidizing agents (present in the whitening agent) from reaching the underlying hard tissue. As a result, some whitening procedures call for pellicle damaging agents and procedures to be applied prior to the application of the whitening agent. Instead, the laser treatment removes the pellicle (and other biofilms if present) allowing the whitening agent to be applied directly to the dental hard tissue undergoing whitening (e.g., enamel, dentine, or cementum). In some cases, direct application of the whitening agent to the dental hard tissue allows the whitening agent to have a reduced dosage (e.g., reducing concentration or reduced quantity of whitening agent). In some embodiments, the whitening agent comprises one or more of hydrogen peroxide, carbamide peroxide, and sodium perborate.

In some embodiments, method 500 is repeated periodically. This is because, the laser treatment typically only affects an outer portion (e.g., thickness no more than about 500 μm, 100 μm, 50 μm, 10 μm, 5 μm, or 2 μm) of the treated surface. This outer portion possesses improved mechanical properties and results in a slowing of the processes associated with enamel aging. However, the outer treated portion is finite and will eventually succumb over time. Additionally, in some cases, a rewhitening will be performed repeatedly until a desired effect has been reached. For this reason, in some cases, it is necessary to repeat treatment periodically. In some cases, it is advantageous to repeat treatment at least once every 10, 5, 3, 2, 1, or 0.5 years. The literature, which describes the vast potential of laser treatment to prevent caries, fails to suggest that periodic retreatment may be necessary and instead tends to assume that a one-time treatment will suffice for most patients. Likewise, no known literature exists which describes the advantageous aspect of synergistic laser treatment and whitening. Additional disclosure related to laser treatment may be found in International Application No. PCT/US 21/15567, by C. Dresser et al., entitled “PREVENTATIVE DENTAL HARD TISSUE LASER TREATMENT SYSTEMS, METHODS, AND COMPUTER-READABLE MEDIA,” the entirety of which is incorporated herein by reference.

To aid in practice of the claimed invention and parameter selection a table is provided below with exemplary ranges and nominal values for relevant parameters.

| | | | |
|---|---|---|---|
| Parameter | Min. | Max. | Nom. |

| | | | | | | |
|---|---|---|---|---|---|---|
| Repetition Rate | 1 | Hz | 100 | KHz | 1 | KHz |
| Pulse Energy | 1 | μJ | 10 | J | 10 | mJ |
| Focal Region Width | 1 | μm | 10 | mm | 1 | mm |
| Fluence | 0.01 | J/cm² | 1 | MJ/cm² | 1 | J/cm² |
| Wavelength | 200-500 | nm | 4000-12000 | nm | 10.6 | μm |

| | | | |
|---|---|---|---|
| Numerical Aperture | 0.00001 | 0.5 | 0.01 |

| (NA) | | | | | | |
|---|---|---|---|---|---|---|
| Focal length | 10 | mm | 1000 | mm | 200 | mm |
| Average Power | 1 | mW | 100 | W | 1 | W |
| Peak Power | 50 | mW | 5000 | W | 500 | W |
| Scan Speed | 0.001 | mm/S | 10 | mm/S | 100,000 | mm/S |

| | | | |
|---|---|---|---|
| Scan Location | 0 | 0.5 × Focal Region | 10 × Focal Region |

| | | |
|---|---|---|
| Spacing | Width | Width |

| | | | |
|---|---|---|---|
| Number of LEDs | 1 | 1000 | 20 |

| | | | | | | |
|---|---|---|---|---|---|---|
| Photoactivating | 0.00001 | W/cm² | 5 | W/cm² | 0.0005 | W/cm² |
| Light Source | | | | | | |
| Irradiance | | | | | | |
| Prescribed | 1 | minute | 12 | hours | 1 | hour |
| Whitening Duration | | | | | | |
| Whitening Time | 0 | minutes | 48 | hours | 30 | minutes |
| Post-Laser | | | | | | |
| Treatment | | | | | | |

| | | | |
|---|---|---|---|
| Whitening Agent | 0.1% | 75% | 12.5% |

Solution
Concentration

| | |
|---|---|
| Photoactivating | 250 nm, 255 nm, 260 nm, 275 nm, 280 nm, 285 nm, 290 nm, 295 nm, |
| Light Source | 300 nm, 310 nm, 325 nm, 340 nm, 375 nm, 385 nm, 395 nm, 405 nm, |
| Wavelengths | 430 nm, 450 nm, 465 nm, 470 nm, 490 nm, 505 nm, 525 nm, 545 nm, |
| | 562 nm, 570 nm, 590 nm, 595 nm, 600 nm, 610 nm, 625 nm, 630 nm, |
| | 635 nm, 639 nm, 645 nm, 660 nm, 670 nm, 680 nm,750 nm, 760 nm, |
| | 770 nm, 780 nm, 810 nm, 830 nm, 840 nm, 850 nm, 870 nm, 890 nm, |
| | 910 nm, 930 nm, 940 nm, 970 nm, 1050 nm, and the like. |
| Whitening Agents | Hydrogen Peroxide, Carbamide Peroxide, Sodium Perborate |

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

FIG. 5 shows a diagrammatic representation of one embodiment of a computing device in the exemplary form of a computer system 500 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure.

Computer system 500 includes a processor 504 and a memory 508 that communicate with each other, and with other components, via a bus 512. Bus 512 may include any of several types of bus structures including, but not limited to, a memory bus, a memory controller, a peripheral bus, a local bus, and any combinations thereof, using any of a variety of bus architectures.

Processor 504 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 504 may be organized according to Von Neumann and/or Harvard architecture as a non-limiting example. Processor 504 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC).

Memory 508 may include various components (e.g., machine-readable media) including, but not limited to, a random-access memory component, a read only component, and any combinations thereof. In one example, a basic input/output system 516 (BIOS), including basic routines that help to transfer information between elements within computer system 500, such as during start-up, may be stored in memory 508. Memory 508 may also include (e.g., stored on one or more machine-readable media) instructions (e.g., software) 520 embodying any one or more of the aspects and/or methodologies of the present disclosure. In another example, memory 508 may further include any number of program modules including, but not limited to, an operating system, one or more application programs, other program modules, program data, and any combinations thereof.

Computer system 500 may also include a storage device 524. Examples of a storage device (e.g., storage device 524) include, but are not limited to, a hard disk drive, a magnetic disk drive, an optical disc drive in combination with an optical medium, a solid-state memory device, and any combinations thereof. Storage device 524 may be connected to bus 512 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 524 (or one or more components thereof) may be removably interfaced with computer system 500 (e.g., via an external port connector (not shown)). Particularly, storage device 524 and an associated machine-readable medium 528 may provide nonvolatile and/or volatile storage of machine-readable instructions, data structures, program modules, and/or other data for computer system 500. In one example, software 520 may reside, completely or partially, within machine-readable medium 528. In another example, software 520 may reside, completely or partially, within processor 504.

Computer system 500 may also include an input device 532. In one example, a user of computer system 500 may enter commands and/or other information into computer system 500 via input device 532. Examples of an input device 532 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 532 may be interfaced to bus 512 via any of a variety of interfaces (not shown) including, but not limited to, a serial interface, a parallel interface, a game port, a USB interface, a FIREWIRE interface, a direct interface to bus 512, and any combinations thereof. Input device 532 may include a touch screen interface that may be a part of or separate from display 536, discussed further below. Input device 532 may be utilized as a user selection device for selecting one or more graphical representations in a graphical interface as described above.

A user may also input commands and/or other information to computer system 500 via storage device 524 (e.g., a removable disk drive, a flash drive, etc.) and/or network interface device 540. A network interface device, such as network interface device 540, may be utilized for connecting computer system 500 to one or more of a variety of networks, such as network 544, and one or more remote devices 548 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 544, may employ a wired and/or a wireless mode of communication. In general, any network topology may be used. Information (e.g., data, software 520, etc.) may be communicated to and/or from computer system 500 via network interface device 540.

Computer system 500 may further include a video display adapter 552 for communicating a displayable image to a display device, such as display device 536. Examples of a display device include, but are not limited to, a liquid crystal display (LCD), a cathode ray tube (CRT), a plasma display, a light emitting diode (LED) display, and any combinations thereof. Display adapter 552 and display device 536 may be utilized in combination with processor 504 to provide graphical representations of aspects of the present disclosure. In addition to a display device, computer system 500 may include one or more other peripheral output devices including, but not limited to, an audio speaker, a printer, and any combinations thereof. Such peripheral output devices may be connected to bus 512 via a peripheral interface 556. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. For example, in some embodiments, fluoride treatment is omitted after laser treatment. Also, features described with respect to certain configurations may be combined in various other configurations. Different aspects and elements of the configurations may be combined in a similar manner. Technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.
* Featherstone, J. D. B., Barrett-Vespone, N. A. Fried, D., Kantorowitz, Z., & Seka, W. (1998). CO₂ laser inhibition of artificial caries-like lesion progression in dental enamel. Journal dental research, 77(6), 1397-1403.
† Featherstone, J, D., & Fried, D. (2001). Fundamental Interactions of Lasers with Dental Hard Tissues. Medical laser application, 16(3), 181-194.
‡ Hsu, D. J., Darling, C. L., Lachica, M. M., &. Fried, D. (2008). Nondestructive assessment of the inhibition of enamel demineralization by CO2 laser treatment using polarization sensitive optical coherence tomography. Journal of biomedical optics, 13(5), 054027.
§ Featherstone, J. D., & Fried, D. (2001). Fundamental Interactions of Lasers with Dental Hard Tissues. Medical laser application, 16(3), 181-194.
** Featherstone, J. D., & Fried, D. (2001). Fundamental Interactions of Lasers with Dental Hard Tissues. Medical laser application, 16(3), 181-194.
†† Vitruk, P. (2014). Oral soft tissue laser ablative and coagulative efficiencies spectra. Implant Practice US, 7(6), 19-27.
‡‡ Zuerlein, M. J., Fried, D., & Featherstone, J. D. (1999). Modeling the modification depth of carbon dioxide laser-treated dental enamel. Lasers in Surgery and Medicine: The Official Journal of the American Society for Laser Medicine and Surgery, 25(4), 335-347.
§§ Xu, C., Reed, R., Gorski, J. P., Wang, Y., & Walker, M. P. (2012). The distribution of carbonate in enamel and its correlation with structure and mechanical properties. Journal of materials science, 47(23), 8035-8043.
*** Featherstone, J. D. B., & Lussi, A. (2006). Understanding the chemistry of dental erosion. In Dental erosion (Vol. 20, pp. 66-76), Karger Publishers.

What is claimed is:

1. A system for synergistic laser cleaning and whitening of teeth, the system comprising:
a laser system configured to perform a laser treatment on exposed tooth surfaces on a plurality of a patient's teeth, the laser system comprising:
a laser arrangement configured to generate a laser beam;
an optical arrangement configured to direct the laser beam toward the exposed tooth surfaces; and
a laser controller configured to control the laser beam to remove a pellicle from the exposed tooth surfaces; and
a tray configured to apply a whitening agent to the exposed tooth surfaces after the laser treatment.

2. The system of claim 1, wherein the tray further comprises a light source configured to illuminate the exposed tooth surfaces for a prescribed whitening duration.

3. The system of claim 3, wherein the prescribed whitening duration is no greater than 1 hour.

4. The system of claim 3, wherein the prescribed whitening duration is within a range of about 0.5 hours to about 3 hours.

5. The system of claim 2, wherein the light source has a wavelength in a range of about 250 nm to about 750 nm.

6. The system of claim 2, wherein the tray comprises a battery configured to power the light source.

7. The system of claim 6, wherein the battery is a zinc air type battery.

8. The system of claim 2, wherein the tray comprises a wireless network connection.

9. The system of claim 1, wherein the whitening agent comprises at least one of carbamide peroxide and hydrogen peroxide.

10. The system of claim 1, wherein the whitening agent has an active ingredient concentration percentage of between 2% and 50%.

11. A method of synergistic laser cleaning and whitening of teeth, the method comprising:
performing, using a laser system, a laser treatment on exposed tooth surfaces on a plurality of a patient's teeth, wherein the laser treatment comprises:
generating, using a laser arrangement, a laser beam;
directing, using an optical arrangement, the laser beam toward the exposed tooth surfaces; and
controlling, using a laser controller, the laser beam to remove a pellicle from the exposed tooth surfaces;
applying, using a tray, a whitening agent to the exposed tooth surfaces within a certain post-laser time after the laser treatment after the pellicle has been removed; and
maintaining, using the tray, application of the whitening agent to the exposed tooth surfaces for a prescribed whitening duration.

12. The method of claim 11, wherein the certain post-laser time is no greater than 1 hour.

13. The method of claim 11, wherein applying the whitening agent, further comprising:
Inserting, using a dispenser, the whitening composition into the tray; and
placing the tray over the exposed tooth surfaces for the prescribed whitening duration.

14. The method of claim 13, wherein the tray comprises a light source having a wavelength in a range of about 250 nm to about 7500 nm.

15. The method of claim 14, wherein the tray comprises a battery configured to power the light source.

16. The method of claim 15, wherein the battery is a zinc air type battery.

17. The method of claim 13, wherein the illumination tray comprises a wireless network connection.

18. The method of claim 11, wherein the prescribed whitening duration is within a range of about 0.5 hours to about 3 hours.

19. The method of claim 11, wherein the whitening agent comprises at least one of carbamide peroxide and hydrogen peroxide.

20. The method of claim 11, wherein the whitening agent has an active ingredient concentration percentage of between 2% and 50%.
